(19) United States
(12) Plant Patent Application Publication
(10) Pub. No.: US 2016/0088781 P1
(43) Pub. Date: Mar. 24, 2016

(54) Almond Tree, 'LONE STAR'

(50) ;

(71) Applicants: John K. Slaughter, Fresno, CA (US); Timothy J. Gerdts, Kingsburg, CA (US)

(72) Inventors: John K. Slaughter, Fresno, CA (US); Timothy J. Gerdts, Kingsburg, CA (US)

(73) Assignee: The Burchell Nursery, Inc., Oakdale, CA (US)

(21) Appl. No.: 14/121,564

(22) Filed: Sep. 18, 2014

(51) Int. Cl. A01H 5/00

(52) U.S. Cl. PLT/155

(57) ABSTRACT

A new and distinct variety of almond tree, which is denominated varietally as ‘Lone Star’, and which produces an attractive, light colored kernel, which is mature for harvesting on approximately August 19 to August 25 under the ecological conditions prevailing in the San Joaquin Valley of central California.

### BOTANICAL DESIGNATION

The present invention relates to a new, novel, and distinct variety of almond tree, Genus ‘Prunus’; Sub-Genus; ‘Amygdalus’ ; Species; Prunus amygdalus Batch.

### VARIETAL DENOMINATION

‘Lone Star’.

### BACKGROUND OF THE NEW VARIETY

The present variety of almond tree resulted from an on-going program of fruit and nut tree breeding. The purpose of this program is to improve the commercial quality of deciduous fruit and nut varieties, and rootstocks, by creating and releasing promising selections of Prunus, Malus, Punica and Juglans species. To this end we make both controlled and hybrid cross pollinations each year in order to produce seedling populations from which improved progenies are evaluated and selected.

The seedling, ‘Lone Star’ was originated by us, and selected from a population of seedlings growing in our experimental orchards which are located near Fowler, Calif. The seedlings, grown on their own roots, were derived from a cross made in early February 1997 using the almond tree ‘Sonora’ (unpatented USDA/U.C. Davis), which was used as the seed parent, and an unnamed almond tree which was used as the pollen parent. After a period of stratification, the seed was placed in the greenhouse by population, and then field planted for tree establishment, and ultimately to produce kernels for evaluation.

One almond seedling from this population, which is the present variety, exhibited especially desirable characteristics, and was then designated as ‘D61.124’. This seedling was marked for subsequent observation. After the 2001 fruiting season, the new variety of almond tree was selected for advanced evaluation and repropagation.

### ASEXUAL REPRODUCTION

Asexual reproduction of this new and distinct variety of almond tree was accomplished by budding the new almond tree onto ‘Nemaguard’ Rootstock (un-patented). This was performed by us in our experimental orchard which is located near Fowler, Calif. Subsequent evaluations of these asexually reproduced plants have shown those asexual reproductions run true to the original tree. All characteristics of the original tree, and its nuts, were established, and appear to be transmitted through these succeeding asexual propagations.

### SUMMARY OF THE VARIETY

‘Lone Star’ is a new and distinct variety of almond tree, which is considered of medium-large size, and which has a moderately vigorous and upright growth characteristic. This new tree is also a regular and productive bearer of relatively large kernels which have a very smooth pellicle, and good eating qualities. This new almond tree has a medium chilling requirement of 450 hours. In addition to the foregoing, the new almond also exhibits self-fertility. The ‘Lone Star’ almond tree bears fruit which are typically ripe for commercial harvesting and shipment on approximately August 19 to August 25 under the ecological conditions prevailing in the San Joaquin Valley of central California. In relative comparison to the ‘Sonora’ almond tree, which is the closest known variety to the variety described herein, the new variety of almond tree bears almonds approximately 5 or more days later; has kernels that are on average 15% larger than that which is produced by the ‘Sonora’ Almond tree, and further the current variety exhibits self-fertility.

### BRIEF DESCRIPTION OF THE DRAWINGS

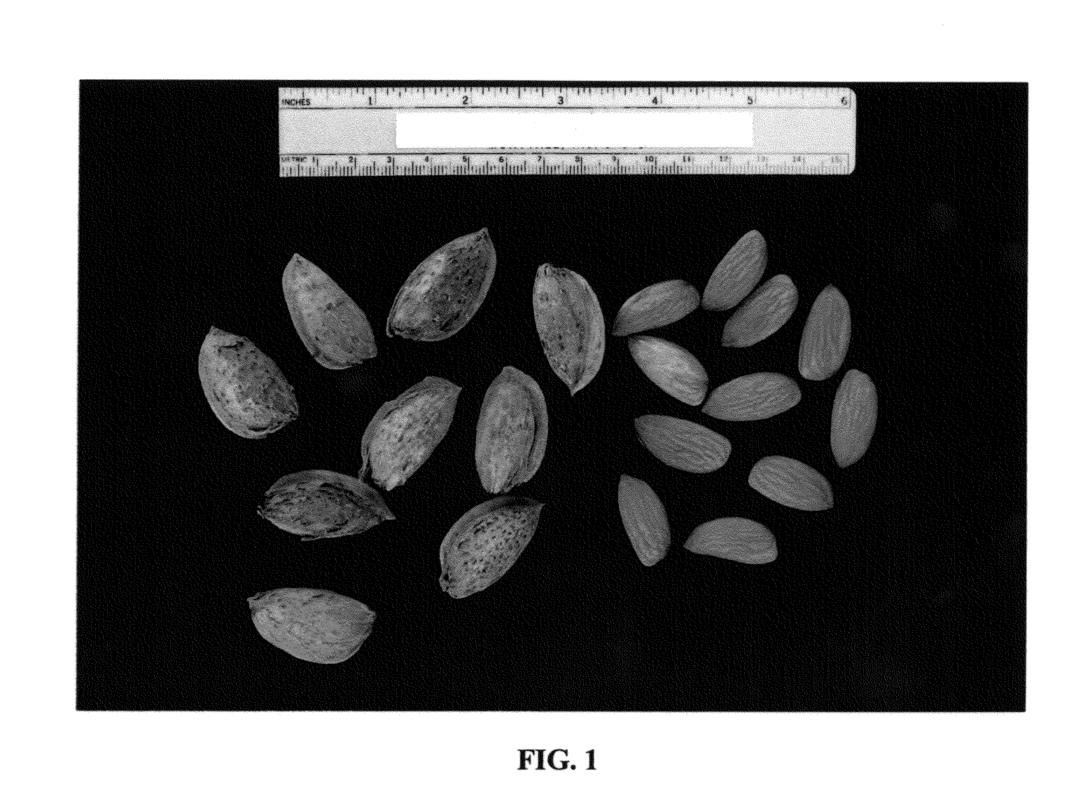

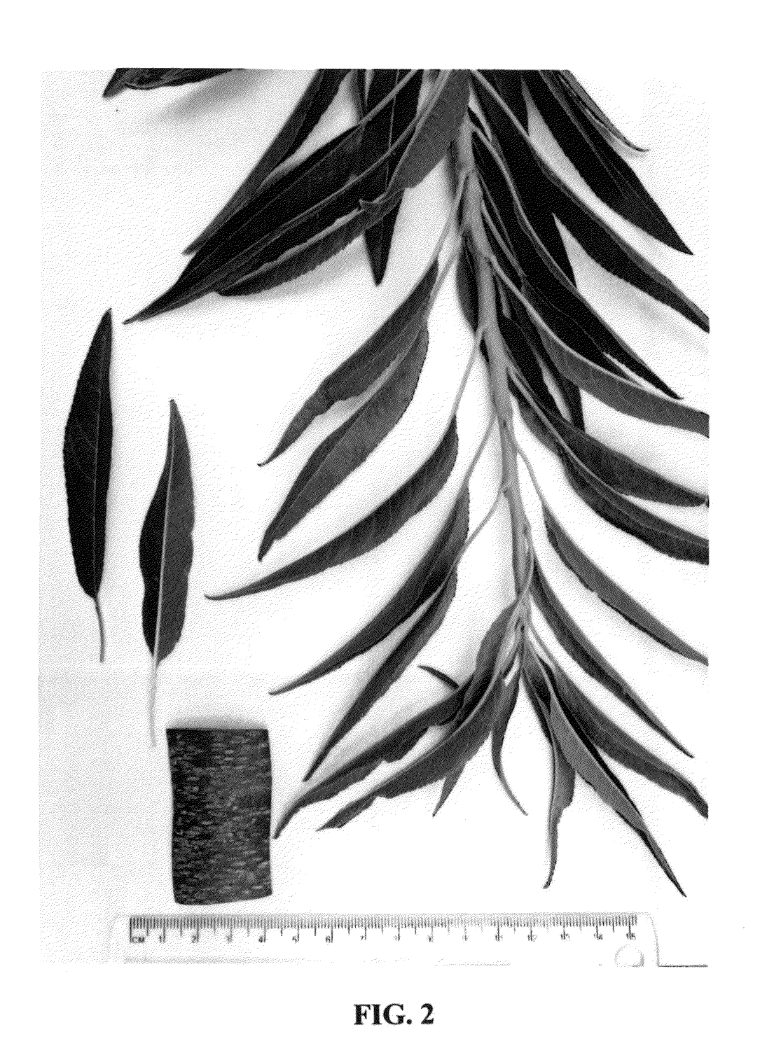

The accompanying drawings, which are provided, are color photographs of the new almond tree variety. FIG. 1 depicts a number of the mature nuts separated from the shell and whole shells containing the nuts inside. This photo is a lateral view and was taken after harvest. FIG. 2 depicts a current season's vegetative shoot, a small segment of limb bark, and two leaves viewed from both the dorsal and ventral perspectives. The colors as seen in these photographs are as nearly true as is reasonably possible in a color representation of this type. Due to chemical development, processing and printing, the leaves and fruit depicted in these photographs may, or may not, be accurate when compared to the actual specimen. For this reason, future color references should be made to the color plates (Royal Horticultural Society, Fourth Edition, 2001) and descriptions provided, hereinafter.

### NOT A COMMERCIAL WARRANTY

The following detailed description has been prepared to solely comply with the provisions of 35 U.S.C. § 112, and does not constitute a commercial warranty, (either expressed or implied), that the present variety will, in the future, display all the botanical, pomological or other characteristics as set forth, hereinafter. Therefore, this disclosure may not be relied upon to support any future legal claims including, but not limited to, breach of warranty of merchantability, or fitness for any particular purpose, or non-infringement which is directed, in whole, or in part, to the present variety.

### DETAILED DESCRIPTION

Referring more specifically to the pomological details of this new and distinct variety of almond tree, the following has been observed during the twelfth fruiting season, and under the ecological conditions prevailing at the orchards of the assignee which are located near the town of Fowler, county of Fresno, state of Calif. All major color code designations are by reference to the R.H.S. Colour Chart (Fourth Edition, 2001), and which is provided by The Royal Horticultural Society of Great Britain. Common color names are also occasionally used.
Tree:
   Size.—Generally considered medium to medium-large, and upright in its growth pattern as compared to other common commercial almond cultivars. The tree of the present variety was pruned to a height of approximately 700.0 cm. to about 730.0 cm. at commercial maturity. It should be understood that tree pruning, canopy development, and the ultimate physical arrangement of almond orchards is effected by a number variables including, but not limited to, the variety chosen, the rootstock on which the new variety is grown, the soil potential, cultural issues, and other agricultural considerations. Therefore, the resulting tree height can be highly variable, and therefore, not necessarily indicative of the current variety.
   Width.—Approximately 655.0 cm.
   Vigor.—Considered moderately vigorous. The present almond tree variety grew from about 200.0 cm. to about 210.0 cm. in height during the first growing season. The new variety was pruned to a height of approximately 150.0 cm. during the first dormant season, and primary scaffolds were then selected for the desired tree structure. Tree vigor can be influenced by a number of variables including soil quality, irrigation practices, nutrition, pruning and nutrition. Consequently, this is not a reliable and distinguishing characteristic of the present variety.
   Productivity.—Productive. When the new variety is grown in a suitable horticultural zone, and under appropriate commercial conditions, the current variety can produce commercial volumes of almonds. The number of the nut set varies with the prevailing climatic conditions; the cultural practices employed, and can be increased with the inclusion and use of active bee hives, and proximity to compatible foreign pollen sources. Although the variety exhibits self-fertility, and is capable of significant yields when planted alone, it has been observed that crop yields increase when the aforementioned practices are observed.
   Fruit bearing.—Regular. Nut set has been more than adequate during the previous 12 years of observation, on both the original seedling, and on subsequent asexually reproduced trees.
   Tree form.—Upright, and typically pruned into a vase shape.
   Density.—Considered moderately dense. It has been determined that pruning the branches from the center of the tree so as to obtain a resulting vase shape, allows for enhanced air movement, and appropriate amounts of sunlight to be received, and which encourages fruit wood development.
   Hardiness.—The present tree was grown and evaluated in USDA Hardiness Zone 9. The calculated winter chilling requirements of the new tree is approximately 450 hours at a temperature below 7.0 degrees C. The present variety of almond tree appears to be hardy under typical central San Joaquin Valley climatic conditions.
Trunk:
   Diameter.—Approximately 28.5 cm in diameter when measured at a distance of approximately 15.24 cm. above the soil level. This measurement was taken at the end of the 12th growing season.
   Bark texture.—Considered moderately rough, with numerous folds of papery scarfskin being present. Since bark development and coloration change with advancing tree age, this characteristic varies with the tree vigor, age and regional conditions. Therefore, this is not a dependable descriptor of the new variety.
   Lenticels.—Numerous flat, oval lenticels are present. The lenticels range in size from approximately 4.0 mm. to about 6.0 mm. in width, and between about 1.0 and about 2.0 mm. in height. The development and size of the trunk lenticels can be influenced, to some degree, by the ambient growing conditions, and are not, necessarily, a dependable characteristic of this variety. As trees of this variety mature, lenticels are present, but they are generally covered by increasing layers of cork (mature bark) and therefore become less apparent.
   Lenticel color.—Considered an orange brown, (RHS Greyed-Orange Group 163 C).
   Bark coloration.—Variable, but it is generally considered to be a greyed brown, (RHS Greyed-Orange Group 166 A). This bark description was taken from trees in their twelfth leaf, and which have ruptured the scarf skin, and which further display deep furrowing as the trunk expands with age. It should be noted that the coloration of the bark can be influenced to some degree by the exposure of the bark to sunlight and humidity.
Branches:
   Size.—Considered medium for the variety.
   Diameter.—Average as compared to other almond varieties. The branches have a diameter of about 22.0 centimeters when measured during the 12th year after grafting.
   Surface texture.—Average, and furrowing of the bark is typically observed by the 4th year of growth.
   Crotch angle.—Primary branches are considered variable, and are usually growing at an angle of about 42 to about 56 degrees when measured from a horizontal plane. This characteristic can be influenced, to some degree, by tree vigor, rootstock, observation time, the degree of pruning, and other cultural conditions effecting the tree.
   Current season shoots.—Surface texture — Substantially glabrous.
   Internode length.—Approximately 15.0 mm to 35.0 mm. Greater internodal intervals exist nearer the basal end of the shoot, and shorter internodal intervals are typically observed at locations nearer to the apical shoot tip.
   Color of mature branches.—Approximately Grey-brown, (RHS Greyed-Orange Group 166 C).
   Current seasons shoots.—Color. — Medium-light green, (RHS Yellow-Green Group 144 C). The color of new shoot tips is considered a bright and shiny green (RHS Yellow-Green Group 145 B). The vegetative shoot color can be significantly influenced by plant nutrition, irrigation practices, and exposure to sunlight, and therefore should not be considered a consistent botanical description of this new variety.
Leaves:
   Size.—Considered medium-large for the species. Leaf measurements have been taken from vigorous, upright, current-season growth, at approximately mid-shoot. It should be understood that the leaf size is often influenced by prevailing growing conditions, quality of sunlight, and the location of the leaf within the tree canopy. For this reason, leaf sizes can vary significantly based upon the amount of ambient light, and other cultural factors listed above, and are not typically considered a dependable botanical descriptor.
   Leaf length.—Approximately 155.0 to about 178.0 mm.
   Leaf width.—Approximately 33.0 to about 36.0 mm.
   Leaf base-shape.—The leaves generally exhibit equal marginal symmetry relative to the leaf longitudinal axis.
   Leaf form.—Lanceolate.
   Leaf tip form.—Acuminate.
   Leaf color.—Upper Leaf Surface — Dark green, (approximately RHS Green Group 137 A).
   Leaf texture.—Glabrous.
   Leaf color.—Lower Leaf Surface — Light to medium green, (approximately RHS Yellow-Green Group 137 C).
   Leaf venation.—Pinnately veined.
   Mid-vein.—Color — Considered a light, yellow-green, (approximately RHS Yellow-Green Group 150 C) in the early, to mid-period, of the growing season.
   Leaf margins.—Considered entire, and smoothly crenate. Occasionally doubly crenate Form. — Considered smooth. Uniformity. — Considered generally uniform.
   Leaf petioles.—Form. — Considered canaliculated, and having a more pronounced trough when viewed from the dorsal aspect. The petiole margin is considered rounded when viewed from the ventral aspect. Size. — Considered medium-large for the species. Length. — About 14.0 to about 33.5 mm. Diameter. — About 1.5 to about 2.0 mm. Color. — Light yellow green, (approximately RHS Yellow-Green Group 149 C).
   Leaf glands.—Size. — Considered very small for the species; approximately 0.5 mm. in length; and about 0.5 mm. in height. Number. — Generally, one, and less common two glands, appear per marginal side are observed. Occasionally, glands are only present on one side. More rarely no classifiable glands are observed. Type. — Glands located at the base of the leaf are predominantly globose in shape.
   Color.—Considered a medium, light brown, approximately (RHS Yellow-Green Group N144 B).
   Leaf stipules.—Size. — Small for this variety. Length. — About 1.0-2.0 mm. Width. — About 0.3-0.5 mm. Number. — Typically 2 per leaf bud, and up to 6 per shoot tip are observed. Form. — Lanceolate, and having a serrated marginal edge. Color. — Green, (approximately RHS Green Group 139 B) when young, but graduating to a brown color, (approximately RHS Greyed-Orange Group 165 A) with advancing senescence. The leaf stipules are generally considered to be early deciduous.
Flower buds:
   Hardiness.—No winter injury (bud death) has been noted during the above-noted years of observation as the variety grew in the central San Joaquin Valley. The new variety of almond tree has not been intentionally subjected to drought, cold or heat stress, and therefore this information is not available.
   Flower bud.—Size — Variable, and dependent upon the state of maturity. The flower buds, as described, were observed approximately 7 days prior to bloom.
   Flower bud.—Length — Approximately 15.0 mm.
   Flower bud.—Diameter — Approximately 10.0 mm.
   Flower bud surface texture.—Pubescent.
   Flower bud orientation.—Considered appressed, but appears less so as the blossoms near opening.
   Bud scale color.—Approximately RHS Greyed-Purple 185 C.
Flowers:
   Date of first bloom.—Observed on Feb. 12, 2014.
   Blooming time.—Considered average in relative comparison to other commercial Almond cultivars grown in the central San Joaquin Valley. The date of full bloom was observed on Feb. 20, 2014. This date of bloom was approximately 4-5days before the ‘Nonpareil’ Almond variety (unpatented). The date of full bloom varies slightly with climatic conditions, and prevailing cultural practices.
   Duration of bloom.—Approximately 8 days. Occasionally 10 days or slightly more bloom time may be observed. This particular characteristic varies slightly with the prevailing climatic conditions.
   Flower class.—Considered a perfect flower, complete and perigynous.
   Flower type.—The variety is considered to have a showy type flower. Petals are largely unappressed relative to the verticle axis of the flower.
   Flower size.—Considered medium large for the species. The flower diameter at full bloom, is approximately 41.0 to 46.0 mm.
   Bloom quantity.—Considered abundant.
   Flower bud frequency.—Generally two flower buds appear per node, occasionally one flower bud per node is observed. Larger numbers of flower buds are present on mature, complex spurs.
   Petal size.—Generally considered medium for the species. Petal Length. — Approximately 17.0 to 20.0 mm. Petal Width. — Approximately 13.0 to 15.0 mm.
   Petal form.—Considered broadly ovate.
   Petal count.—Nearly always 5.
   Petal texture.—Glabrous.
   Petal color.—Considered a light pink at the popcorn stage, (RHS Red Group 56 A), and becoming lighter with increasing senescence to nearly a pure white, (RHS White Group N155 D).
   Fragrance.—Slight.
   Petal claw.—Form. — The claw is considered obovate, and is generally medium-small and appears more elongated. Length. — Approximately 15.0-16.0 mm. Width. — Approximately 7.5 to 9.0 mm.
   Petal margins.—Generally considered variable, from nearly smooth to moderately undulate.
   Petal apex.—Often the petal margin exhibits a shallow, and wide recess, at the petal tip. Petal Apex Width. — Approximately 2.0 mm. Petal Apex Depth. — Approximately 2.0 mm.
   Flower pedicel.—Length. — Considered medium-long with an approximate length of about 3.5 to about 4.5 mm. Diameter. — Approximately 2.5 mm. Color. — A medium brown, approximately (RHS Grey-Brown Group 199 C). This color depends upon the pedicel selected, fruit maturity, and the time of observation. Surface Texture. — Glabrous.
   Floral nectaries.—Color. — Considered a deep reddish orange (approximately RHS Greyed-Red Group 178 D).
   Calyx.—Surface Texture. — Generally glabrous. Color. — A dull red, (approximately RHS Greyed-Red Group 178 C).
   Sepals.—Surface Texture. — The surface has a short, fine pubescent texture. Number. — 5 sepals are observed. Size. — Average, and ovate in form. Sepal Length. — Approximately 4.0 to 6.0 mm. Sepal Width. — Approximately 3.5 to 4.5 mm. Sepal Shape. — Generally obovate. Sepal Margin. — Considered smooth and entire. Sepal Color. — A dull greenish brown, (approximately RHS Yellow-Green Group 152 C).
   Anthers.—Generally. — Average in size. Color. — Red to yellow-gold when viewed dorsally at dehiscence, (approximately RHS Yellow Group 13 B).
   Pollen production.—Pollen is abundant and has a yellow color, (approximately RHS Yellow Group 13 C).
   Fertility.—Self-fertile.
   Filaments.—Size. — Approximately 9.0 to 11.0 mm. in length; and 0.5 to 0.75 mm. in width. Color. — Considered white to a bright white, (RHS White Group 155 C).
   Pistil.—Number. — Nearly always one. Generally. — Medium small in length. Length. — Approximately 11.0 to about 13.5 mm. including the ovary. Color. — Considered a very pale yellow-green, (approximately RHS Greyed Yellow Group 160 C). Surface Texture. — The variety has a pubescent pistil.
Kernel:
   Maturity when described.—Firm, and having a dry pellicle condition. — Approximately Aug. 22, 2013. The date of harvest can vary with the prevailing climatic conditions, crop loads and cultural practices utilized on the trees in question.
   Size.—Generally — Considered large, and very uniform.
   Average kernel length.—Approximately 26.0 to about 30.0 mm.
   Average kernel width.—Approximately 12.0 to about 15.0 mm.
   Average kernel thickness.—Approximately 7.0 to about 11.0 mm. These dimensional characteristics are quite dependent upon the crop load, and the prevailing cultural practices, and therefore is not a particularly distinctive botanical descriptor of the new variety.
   Kernel form.—Generally the respective kernels are typically uniform in shape. As compared to the ‘Sonora’ variety, the kernels of the present variety are slightly broader and slightly darker in color.
   Kernel.—Color. — Considered a light golden brown (RHS Greyed-Orange 164 C) Apex. — Shape — Rounded to slightly retuse.
   Base.—Shape. — Slightly oblique relative to the verticle axis. Thickness. — Considered medium in thickness. Surface Texture. — A short, fine, pubescence is observed. Surface veining is present throughout the pellicle. Color of veins. — Considered a medium brown, approximately (RHS Greyed-Orange 166 C).
   Taste.—Mild, pleasant, non astringent.
   Kernel weight.—Approximately 1.35 grams per kernel although the weights of kernels can be highly affected by climatic conditions and prevailing cultural practices. Consequently, it is possible to observe kernels with both higher and lower weights.
   Kernel texture.—Firm, and dense.
   Kernel aroma.—Not apparent
   Eating quality.—Considered very good.
Shell:
   Size.—It is generally considered to be medium-large for the species. The shell size varies significantly depending upon the tree vigor, the crop load, and the prevailing growing and cultural conditions under which the tree was grown.
   Length.—Average, about 30.0 to about 39.0 mm.
   Width.—Average, about 19.0 to about 22.0 mm.
   Thickness.—Average, about 11.0 to about 15.0 mm.
   Form.—Roughly ovoid.
   Shell base.—Shape — The stone is considered shortly attenuate.
   Apex.—Shape — The shell exhibits a slight to prominently cuspinate apex.
   Shell surface texture.—Considered reasonably smooth with considerable pitting and almost nonexistent furrowing or ridging is observed. Ventral Edge. — The ventral edge generally exhibits a thin, fine, and protruding fin at the sutural margin. Dorsal Edge. — Shape — Generally considered even. The folds of the surface ridges which appear on the external margins often end gently along the suture. Shell Color. — The color of a mature, dry stone is generally considered a dull brown, approximately (RHS Greyed-Orange Group 164 B).
   Use.—The present variety ‘Lone Star’ is considered to be an almond tree of the early mid-season of maturity, and which produces kernels that are useful in various almond categories and are further blanchable.
   Keeping quality.—Appears excellent.
   Resistance to insects and disease.—No particular susceptibilities were noted. The present variety has not been intentionally tested to expose or detect any susceptibilities or resistances to any known plant, fruit diseases, insect, frost, winter injury or other environmental factors.

Although the new variety of almond tree possesses the described characteristics when grown under the ecological conditions prevailing near Fowler, Calif., in the Central part of the San Joaquin Valley of California, it should be understood that variations of the usual magnitude, and characteristics incident to changes in growing conditions, fertilization, nutrition, pruning, pest control, frost, climatic variables and changes in horticultural management are to be expected.

Having thus described and illustrated our new variety of almond tree, what we claim is new, and desire to secure by plant Letters Patent is:

1. A new distinct variety of almond tree, substantially as illustrated and described, and which is characterized principally as to novelty by being self-fertile and producing an attractively, light colored kernel which is mature for harvesting on approximately August 19 to August 25 under the ecological conditions prevailing in the San Joaquin Valley of central California.
